(19) United States
(12) Patent Application Publication
(10) Pub. No.: US 2012/0095428 A1
(43) Pub. Date: Apr. 19, 2012

(54) MULTIPAGE BOOK WITH ABSORBENT PAGES

(76) Inventor: Leslie Ann UKE, Del Mar, CA (US)

(21) Appl. No.: 13/088,356

(22) Filed: Apr. 16, 2011

Related U.S. Application Data
(60) Provisional application No. 61/325,258, filed on Apr. 16, 2010.

(51) Int. Cl. A61F 13/45; B32B 3/00

(52) U.S. Cl. 604/385.15; 428/57

(57) ABSTRACT

Articles that each comprise a binding of a plurality (i.e., at least two) of absorbent, reusable, washable sheets that can be independently accessed by a user when needed to absorb a liquid-containing substance are described, as are methods of such articles, or sheets therefrom, to perform various methods, including absorbing liquids.

### BACKGROUND OF THE INVENTION

1. Field of the Invention

The present invention relates generally to articles useful for absorbing fluids.

2. Background

The following description includes information that may be useful in understanding the present invention. It is not an admission that any such information is prior art, or relevant, to the presently claimed inventions, or that any publication specifically or implicitly referenced is prior art.

Parents and caregivers are quite familiar with spilled drinks, runny noses, and other occurrences that require the removal of fluids. Many solutions have been devised or used, including fabric rags and towels, face cloths, handkerchiefs, Kleenex, paper towels, and the like. Some of these products are reusable, and are typically washed between uses, while others, such as Kleenex, paper towels, and disposable wipes, are discarded after use. Despite these existing solutions, there remains a long-standing unmet need for improved, environmentally sensitive absorbent articles. This invention addresses that need.

### Definitions

When used in this specification, the following terms will be defined as provided below unless otherwise stated. All other terminology used herein will be defined with respect to its usage in the particular art to which it pertains unless otherwise noted.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

By “removable” or “removably” is meant detachable in a way that does not damage the item removed or the article from which the item is removed. Also, a “removable item”, for example, an absorbent, washable sheet, may also be re-attached or otherwise re-associated with the article from which it was removed.

### SUMMARY OF THE INVENTION

The object of this invention is to provide a new, patentable class of articles useful for absorbing liquids, including spilled liquids and bodily fluids (e.g., nasal secretions, tears, mucous, phlegm, sputum, and urine). Thus, one aspect of the invention relates to articles that each comprise a binding of a plurality (i.e., at least two) of absorbent, reusable, washable sheets that can be independently accessed by a user when needed to absorb a liquid-containing substance. In such articles the sheets, or pages, are bound in a “book configuration”, i.e., a configuration wherein the plurality of sheets are fastened together to produce an article that, in preferred embodiments, resembles a book-like product. In an article according to the invention, the absorbent sheets resemble “pages”, and these sheets can be used to absorb liquids and liquid-containing materials and substances, to clean surfaces, to spread materials, and/or to release compositions stored in a sheet. The bound product may or may not include a cover material to protect the absorbent sheets from the environment and/or to prevent unintended release of compositions stored in one or more of the “book's” “pages”. The absorbent sheets can be made of any suitable synthetic or natural absorbent materials, or combinations of synthetic or natural materials. One or more non-absorbent dividers or barriers may also be included in the binding so as to prevent liquid absorbed into one sheet from flowing or otherwise being transported into an adjacent sheet when the book is closed. Barriers or dividers may be separate, non-attached insertable members or they may be bound into the book. When bound, preferably they are not removable, although in some embodiments, they may be.

Any suitable book binding technique can be used to assemble to the absorbent sheets, along with cover materials, non-absorbent barriers, etc., into a book-like article according to the invention. As will be appreciated, the removable absorbent sheets can be permanently secured in a book, or they may be removable or detachable. If one or more sheets are detachable, the book further comprises a complementary page retainer for each such page, from which the page can be detachably removed before or after use and then reattached when desired (for example, after cleaning) Any page retainer will remain secured in the book when its corresponding page is removed.

Books of the invention can be of any desired size, and may contain anywhere from 2-1,000 or more absorbent sheets, although embodiments having 2-200, preferably 2-50, sheets, are preferred.

A related aspect of the invention relates to methods of using the articles of the invention, for example, to absorb liquids from various surfaces, to clean surfaces, to spread materials or compositions on surfaces, and to deliver materials or compositions stored in the sheets.

These and other aspects and embodiments of the invention are discussed in greater detail in the sections that follow. The foregoing and other aspects of the invention will become more apparent from the following detailed description, accompanying drawings, and the claims. The descriptions and drawings that follow are illustrative only and not intended to be limiting.

### BRIEF DESCRIPTION OF THE DRAWINGS

A brief summary of each of the figures is provided below.

FIG. 1 shows several embodiments of articles according to the invention.

FIG. 2 shows several embodiments of articles according to the invention that have removable sheets.

FIG. 3 shows several embodiments of articles according to the invention that include pouches holding a container that contains a solution.

FIG. 4 shows two alternative embodiments of articles according to the invention.

### DETAILED DESCRIPTION

The following descriptions refer to the appended drawings, which illustrate representative embodiments of the articles and methods of the invention. In the drawings, like numbers represent like elements.

Several labeled figures are also included with this application to show representative embodiments of the inventions. As will be seen from the appended drawings, many representative embodiments of the invention relate to a book that comprises a plurality of absorbent fabric pages, wherein each page is bound into the book along one edge of the page's periphery. In some cases one or more of the pages are removable. In the embodiments shown in several of the appended drawings, the book also includes front and back covers to protect the absorbent pages. In some embodiments, different types of optional closures are also depicted, including buttons, zippers, Velcro, and snaps. Books can also include non-absorbent sheets between the absorbent pages. In this way, after a sheet has been used to absorb liquid, movement of some of that liquid to neighboring absorbent sheets (or to a front or back cover) can be prevented. In such embodiments, any natural or synthetic material that can serve as a barrier, preferably an impermeable barrier, to fluid movement can be employed. Plastics and rubber, or fabrics impregnated or covered with plastics and/or rubber, are preferred as barrier sheets.

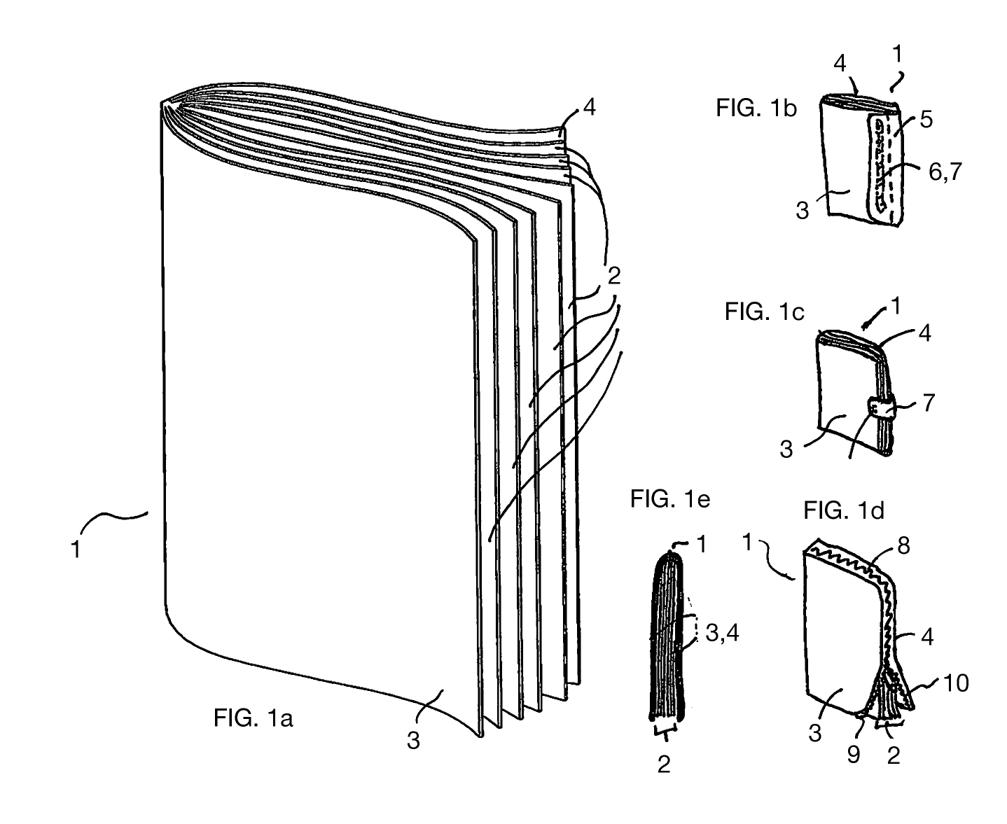

Turning now to the drawings, FIG. 1 shows several embodiments of the invention, labeled A-D. Drawing 1.E shows a top down view of an article of the invention. Drawing 1.A shows a book (1) containing a plurality of absorbent, reusable, washable sheets (2) that can be independently accessed by a user. The sheets can be made from any suitable absorbent, reusable, washable material. Absorbent, washable fabrics are particularly preferred. These fabrics can be made from synthetic or natural materials, including cotton (with organic cotton being particularly preferred), as well as blends of natural and synthetic materials. The sheets are bound into the book using any suitable binding technique, including stitching and adhesives. Preferably, the book has a binding, and includes a front and back covers (3, 4), which may or may not be absorbent as well. In some embodiments, the front and back covers (3, 4) are also sheets (2), and can themselves be used to absorb liquids.

Sheets in such a book can be used to absorb liquids of various types. For example, a sheet can be used to blow one's nose, as one would use a handkerchief. As will be appreciated, when used for this and similar purposes, the multiple bound fabric pages allow a more sanitary way of keeping nasal secretions (or other bodily fluids) from contaminating the surrounding environment, be it hands, pockets, or interiors of purses, diaper bags, and the like. The book is reusable and washable, and will help to reduce waste and unnecessary use of resources such as trees and water.

FIG. 1.B-D show three embodiments of a book (1) that can be closed. In FIG. 1.B, the rear cover (4) of the book (1) has an extended, foldable front portion (5) that, when the book is in the closed position, can be wrapped around to overlap with the front portion of the front cover (3). In the embodiment shown, the inside of the extended, foldable front portion (5) has a Velcro strip (6) that mates to a complementary Velcro strip disposed on the outside of the front portion of the front cover (3). In FIG. 1.C, the rear cover (4) of the book (1) has an extended, foldable front tab (7) that, when the book is in the closed position, can be wrapped around to overlap with and engage the front portion of the front cover (3). In this embodiment, the extended, foldable front tab (7) has a button hole through which a button (8) attached to the front portion of the front cover (3) can be used to fasten the flap (3) in order to keep the book (1) closed until the user desires to open it and expose the bound sheets (2), any one or more which can then be used to absorb liquid by bringing the sheet into contact with the liquid to be absorbed. In other similar embodiments that are not illustrated, the inside of the extended, foldable front tab could, for example, have a snap-type retainer adapted to engage a complementary retaining structure disposed on the outside of the front portion of the front cover. FIG. 1.D illustrates an embodiment wherein the book (1) has front and rear covers (3,4) that can be zipped together using a zipper. Portions of the periphery of the front and rear covers (3, 4) that can be brought into proximity are equipped with complementary zipper components (9, 10).

The invention also includes embodiments wherein sheets or “pages” of the book are removable, and may or may not be disposable. In the context of removable pages, they are secured into the article using any suitable attachment configuration, for example, Velcro strips, snaps, interlocking flanges and channels, etc. Alternatively, one or more releasable connectors (e.g., key rings) can be employed to bind sheets, as well as covers, dividers, and such other elements as may be included in a particular embodiment.

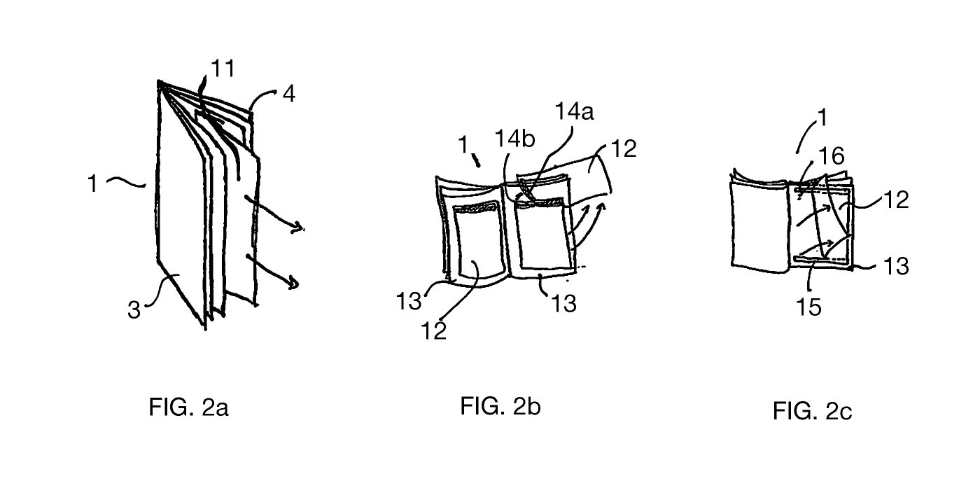

FIG. 2 shows three embodiments, A, B, and C, of books (1) that contain at least one removable page (11). In drawing 2.A, the removable, absorbent, washable sheet (1) can be removed from the book. Any suitable feature that allows for easy removal and reattachment of a removable sheet (11) can be used in the book. In drawings 2.B and 2.C, a removable sheet (12) can be removed from a carrier page (13), which may carry removable sheets (12) on both or just one side of the carrier page. Drawing 2.B shows the removable sheet (12) carried by the carrier page (13) on right being removed from the book by lifting the page so as to disengage the Velcro components (14a, 14b) disposed on the carrier page (13) and removable sheet (12). In the embodiment shown in drawing 2.C, the removable sheet (12) is secured to the carrier page (13) at both the top and bottom using any suitable retention system (e.g., Velcro, 15, 16)

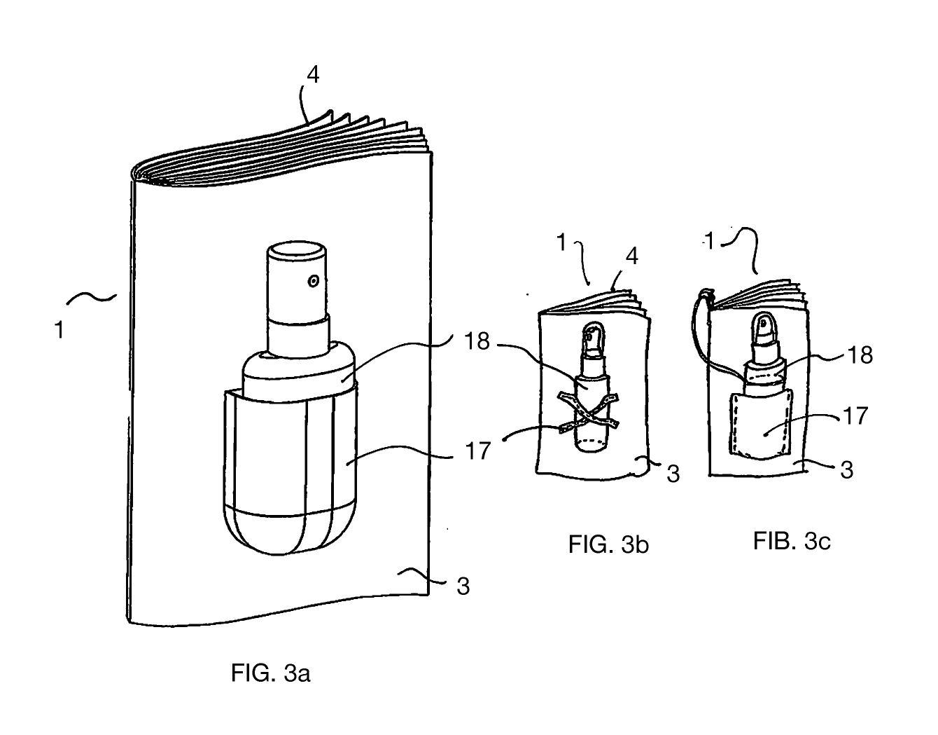

FIG. 3 shows three embodiments of a book according to the invention that further includes one or more retainers or pouches (17) for holding a container (18), for example, a spray bottle that may be filled, for example, with a cleaning solution. Of course, other items can also be carried in the retainer, for example, bottles or tubes of sunscreen. A retainer (18) is preferably disposed on the front or back cover of the book (1).

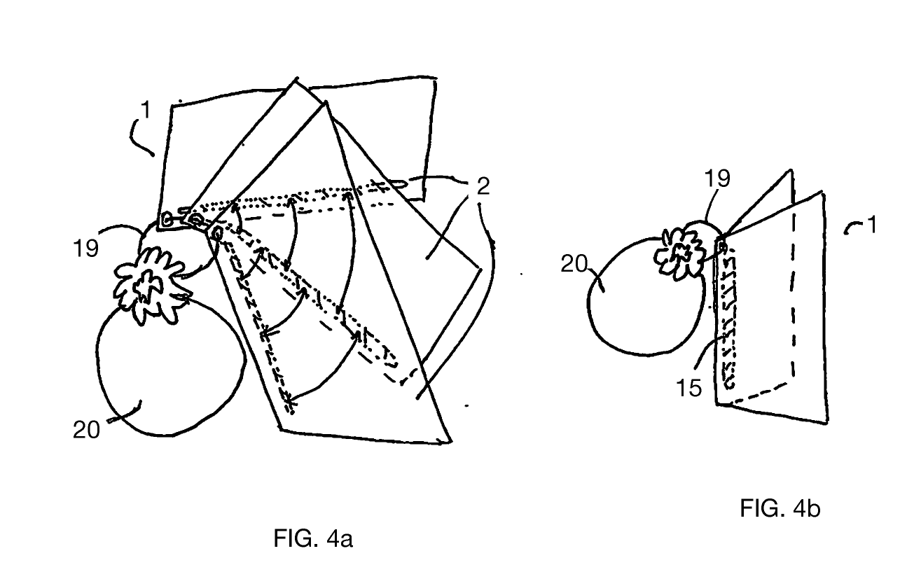

Two drawings, drawings 4.A and 4.B in FIG. 4, also depict articles according to the invention (1) wherein the pages (2) are bound using a ring (19) that allows the pages to be associated via hole near one corner of each page and cover. In these embodiments, a storage container (20; here, a small bag having a drawstring closure) is also included for storage of absorbent sheets after they have been removed and used. At some point the used sheets can be removed from the storage bag and cleaned, for example, by laundering, after which they can re-associated with one or more other like pages in order to reconstitute an article containing clean pages. As will be appreciated, this process can be iterated multiple times.

Those in the art will understand that the articles of the invention can be used for many different applications, including to absorb liquids from various surfaces, as described above, as well as to clean surfaces, to spread materials or compositions on surfaces, and to deliver materials or compositions stored in the sheets. Several of such uses are described in more detail below.

Use as a cleaning wipe: A sheet from a book according to the invention can be used to clean various surfaces, typically by absorption of a liquid, for example, a spilled drink or a bodily fluid such as sinus drip, onto one or more pages in or removed from a book according to the invention. By having multiple bound pages, multiple cleanings can be performed, preferably each with clean sheet, although if necessary a single sheet may be used more than once between cleanings. The number of pages and type of fabric used to make a particular book is left to the discretion or the ordinarily skilled artisan. As will be understood, in some cases it will be desirable to use a sheet adapted for a particular application (e.g., an absorbent sheet that contains a cleaning solution absorbed therein for use to clean a surface (e.g., skin, a countertop or tabletop, etc.) by wiping with the sheet), while in other embodiments the sheet can be a standard material suitable for use in many applications. As will be appreciated, one or more pages can be used in conjunction with an externally applied composition (applied directly to the sheet or onto another surface prior to contact with the sheet) in a manner analogous to how one might use a rag (e.g., for polishing, applying cleaning solution to surfaces, etc.). As will be appreciated, books that further include a pouch, pocket, or other structure configured to hold or be connected to one or more containers that contain a composition to be applied using a sheet from the book will be particularly useful in the context of methods that include application or use of solution or other composition to the surface of an object.

Use for application of liquid, gel, and paste compositions and the like: Here, an article of the invention is used for applying to a target surface a liquid, gel, or paste composition previously absorbed into a sheet of the book. The composition is released from the sheet upon physical contact or chemical exposure to the target surface. If desired, in these embodiments a protective liner or divider can be used to enclose or physically partition individual pages so as to keep the formula active and/or protect the formula from spreading, becoming contaminated, or being absorbed by adjacent pages.

Combination Uses: The invention also includes embodiments wherein different sheets within a particular book are configured for different uses. For example, some pages can be used for absorbing, some for applying compositions, and some for releasing compositions stored or loaded into a particular sheet for subsequent use.

Although the invention has been described with reference to the above Detailed Description, it will be understood that modifications and variations are encompassed within the spirit and scope of the invention. Accordingly, the invention is limited only by the appended claims.

All of the articles and methods described and claimed herein can be made and executed without undue experimentation in light of the present disclosure. While the articles and methods of this invention have been described in terms of preferred embodiments, it will be apparent to those of skill in the art that variations may be applied to the articles and methods and in the steps or in the sequence of steps of the method described herein without departing from the spirit and scope of the invention as defined by the appended claims.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

I claim:

1. A method, comprising:
a. providing an article comprising a plurality of absorbent, reusable, washable, optionally removable sheets bound in a book configuration;
b. using at least one sheet from the article to absorb a liquid, wherein optionally at least one sheet is removable from the article and optionally wherein at least one of the removable sheets is first removed from the article before the sheet is used to absorb the liquid, wherein the liquid is optionally a spilled liquid or a bodily fluid; and
c. cleaning the article or the removable sheet, optionally by laundering the article, after the article or removable sheet is used to absorb the liquid.

2. An article comprising a plurality of absorbent, reusable, washable sheets bound in a book configuration.

3. An article according to claim 2 that comprises from 2-200 absorbent, reusable, washable sheets.

4. An article according to claim 2 wherein the washable sheets are permanently or removably bound in or to the article.

5. An article according to claim 2 that further comprises at least one barrier sheet.
